(19) United States
(12) Patent Application Publication
(10) Pub. No.: US 2024/0100193 A1
(43) Pub. Date: Mar. 28, 2024

(54) Self-complementary AAV vectors carrying dominant negative RhoA and methods of use to treat ocular diseases

(71) Applicant: iView Therapeutics, Inc., Cranbury, NJ (US)

(72) Inventors: Terete Borras, Chapel Hill, NC (US); Guiying Hong, Doylestown, PA (US); Bo Liang, Plainsboro, NJ (US); Ling Wu, Fair Lawn, NJ (US); Carola Maturana, Princeton, NJ (US)

(21) Appl. No.: 18/468,299

(22) Filed: Sep. 15, 2023

Related U.S. Application Data
(60) Provisional application No. 63/407,150, filed on Sep. 15, 2022.
(60) Provisional application No. 63/414,996, filed on Oct. 11, 2022.
(60) Provisional application No. 63/426,725, filed on Nov. 19, 2022.

(51) Int. Cl. A61K 48/00; A61K 38/46; A61P 27/06; C12N 15/86

(52) U.S. Cl. CPC A61K 48/0066; A61K 38/46; A61K 48/0041; A61K 48/0075; A61P 27/06; C12N 15/86; C12N 2750/14122; C12N 2750/14143; C12N 2750/14145; C12N 2750/14171

(57) ABSTRACT

Provided herein are recombinant self-complementary adeno-associated virus (scAAV) nucleic acid vectors that comprise a ubiquitous eukaryotic promoter, such as elongation factor 1α (EF1α), chicken beta-actin (CBA), and hybrid chicken beta-actin (CBh), followed by a dominant negative RhoA. Also provided herein are methods of use of said vectors, including intraocular injections (e.g., intracameral injections) to reduce intra-ocular pressure (TOP). Also provided herein are plasmids, recombinant scAAV particles, compositions, formulations, and other methods of use related to such vectors.

### STATEMENT OF PRIORITY

This application claims the benefit of U.S. Provisional Patent Application Ser. No. 63/407,150, filed Sep. 15, 2022; U.S. Provisional Patent Application Ser. No. 63/414,996, filed Oct. 11, 2022; and U.S. Provisional Patent Application Ser. No. 63/426,725, filed Nov. 19, 2022; the disclosure of each of which is incorporated by reference herein in its entirety.

### STATEMENT REGARDING ELECTRONIC FILING OF A SEQUENCE LISTING

A Sequence Listing in XML format, entitled 1635-2_ST26.xml, 16,536 bytes in size, generated on Sep. 13, 2023 and filed herewith, is hereby incorporated by reference in its entirety for its disclosures.

### FIELD OF THE INVENTION

Provided herein are recombinant self-complementary adeno-associated virus (scAAV) nucleic acid vectors that comprise a ubiquitous eukaryotic promoter, such as elongation factor 1α (EF1α), chicken beta-actin (CBA), and hybrid chicken beta-actin (CBh), followed by a dominant negative RhoA. Also provided herein are methods of use of said vectors, including intraocular injections (e.g., intracameral injections) to reduce intra-ocular pressure (TOP). Also provided herein are plasmids, recombinant scAAV particles, compositions, formulations, and other methods of use related to such vectors.

### BACKGROUND OF THE INVENTION

Advances in research and development for gene therapy for eye diseases is leading the trend to bring hope for a cure to more patient groups soon. Traditional gene therapies focus on inherited eye diseases such as retinitis pigmentosa, chloridemia, Leber hereditary optic neuropathy, Leber congenital amaurosis (LCA), color blindness, and X-rays Linked Retinitis Pigmentosa (XLRS), among others. New generations of gene therapies target indications of chronic disease such as wet age-related macular degeneration (AMD), diabetic retinopathy, and other chronic retinal conditions.

Glaucoma is one of the leading causes of blindness in people over the age of 60 and the second leading cause of blindness in the world. In the United States, the prevalence for glaucoma is about 19 out of 1,000 people. This translates to about 22 out of every 1,000 females and 16 out of every 1,000 males (National Eye Institute. Glaucoma tables). The trabecular meshwork (TM) provides the resistance to aqueous humor flow that is needed to maintain a physiological intraocular pressure (TOP). TM dysfunction results in elevated IOP (e.g., ocular hypertension), which is the major risk factor for the development of glaucoma.

Currently, the available treatments in clinics are daily eye drops and surgery to lower IOP. There are several types of eye drops used to treat glaucoma, and they work by different mechanisms to lower IOP. Common eye drops include prostaglandin analogs, beta-blockers, alpha-agonists, carbonic anhydrase inhibitors and rho kinase inhibitors. Most glaucoma patients are elderly individuals who often require additional medications for other diseases, thus compliance of these patients for daily eye drop use is poor.

When eye drop medications cannot lower IOP enough or cause significant side effects, surgery becomes an option. Laser trabeculoplasty is one of the most common surgeries used to treat open-angle glaucoma by helping to improve the outflow of aqueous humor. Although the laser surgery has a very good safety profile, there are still some drawbacks and limitations to be aware of, such as limited duration of effects, variable responses, need for continued monitoring and temporary IOP spike. If the patient's IOP is not controlled with topical eye drops or laser trabeculoplasty, Microinvasive Glaucoma Surgery (MIGS) is suggested. MIGS offers IOP lowering by creating a bypass to normal aqueous outflow mechanisms. Bypass can be achieved by placing a trabecular meshwork bypass stent or goniotomy (or trabeculotomy) that improves aqueous outflow into Schlemm's canal. The risks of these surgeries include variations in efficacy, needs for repeated procedures and long-term durability. Considering the drawbacks and limitation of current treatments, development of a gene therapy regimen to reduce IOP which would allow infrequent (or sole) administrations could bring great benefits to glaucoma patients.

RhoA is a GTP-binding protein known to play a role in cell contractility. RhoA cycles between an active and inactive form, and the cycling between the two conformations activates Rho-associated kinase (ROCK). The relevance of the RhoA pathway in modulating outflow facility was first reported as RhoA mediating cell contraction in the TM. A study showed that the target of a smooth muscle cell contraction inhibitor (Y-27632) was a ROCK inhibitor, and that this inhibitor reduced cell contraction by decreasing calcium sensitivity in the vascular system. These findings, together with the well-stablished role of TM cell contraction in aqueous outflow facility, triggered a myriad of studies which demonstrated that Y-27632 was also influencing IOP. Since then, ROCK inhibitors have been successfully developed as new drugs for glaucoma treatment. However, the fast metabolism of chemical ROCK inhibitors leads to inconveniently frequent doses to achieve a reduction of IOP. To fill the unmet medical need to treat glaucoma, it is thus desirable to develop novel therapies specifically targeting TM tissues and, more specifically, the RhoA pathway to benefit the aged population.

### SUMMARY OF THE INVENTION

The present invention is based on the finding that gene therapies using dominant negative mutation of RhoA (dnRhoA) can lower IOP in glaucoma patients by inhibiting ROCK signaling. Universal expression promoters can be used to drive the expression of the mutated dnRhoA. The present invention further relates to the surprising enhancement of dnRhoA expression when shorter eukaryotic promoters are used. Several AAV capsids, including mutated AAV2, can also be used to package the viral vectors as described.

Thus, one aspect of the invention relates to recombinant self-complementary adeno-associated virus (scAAV) particles comprising a viral capsid protein and a scAAV nucleic acid vector that comprises a eukaryotic promoter and dominant negative RhoA. In some embodiments, the dominant negative RhoA comprises at least one amino acid mutation (e.g., T19N). In some embodiments, the ubiquitous promotor comprises truncated EF1α, CBA, CBh, or other short eukaryotic promoter.

In some embodiments, the recombinant scAAV particles use capsids from different viral serotypes. In some embodiments, the viral capsid comprises one or more amino acid mutation, e.g., capsid with amino acid substitution at one or more positions at Y444F, Y500F, and/or Y730F, numbered according to VP1 of AAV2 (SEQ ID NO:5). In some embodiments, the one or more amino acid mutations in the viral capsid reduce the immunogenicity, increase the expression of the dominant negative RhoA transgene, and/or increase the duration of expression of the dominant negative RhoA transgene.

Also provided herein are methods of reducing the TOP in a subject in need thereof, the method comprising administering a therapeutically effective amount of a recombinant scAAV particle as described herein. In some embodiments, the recombinant scAAV particles can be administered by intraocular injection, optionally wherein the injection is into the anterior chamber of the eye (e.g., to the trabecular meshwork tissue and/or the cornea) and/or into the posterior chamber of the eye (e.g., to the retinal cells, including retinal ganglion cells (RGC) and/or retinal pigmented epithelial cells (RPE)) and/or into the iris.

Another aspect of the invention relates to methods of treating and/or preventing an ocular disease in a subject in need thereof, the method comprising administering a therapeutically effective amount of a recombinant scAAV particle as described herein.

A further aspect of the invention is the use of a recombinant scAAV particle as described herein for reducing the TOP in a subject in need thereof or methods of treating and/or preventing an ocular disease in a subject in need thereof.

An additional aspect of the invention is the use of a recombinant scAAV particle as described herein in the manufacture of a medicament for reducing the TOP in a subject in need thereof or methods of treating and/or preventing an ocular disease in a subject in need thereof.

These and other aspects of the invention are set forth in more detail in the description of the invention below.

### BRIEF DESCRIPTION OF THE DRAWINGS

FIG. 1 is an image showing the schematics of the expression cassettes used in the plasmids.

FIG. 2 is a series of fluorescent microscopy images and associated flow cytometry measurement graphs showing the expression of eGFP from different promoters at 72 hours post transfection in pooled human trabecular meshwork (HTM) cells.

FIG. 3 is a series of fluorescent microscopy images and associated flow cytometry measurement graphs showing the expression of eGFP from different promoters at 72 hours post-transduction (multiplicity of infection 10,000) on pooled HTM cells.

FIG. 4 is a graph showing the quantification of GFP positive cells by flow cytometry after plasmid transfection or viral infection.

FIG. 5 is an image of a Southern blot showing the restriction digestion of the plasmids containing the dnRhoA gene with a PvuII-HF restriction enzyme, viewed on a 1% agarose gel; lane 1 is a 1 kilobase (kb) ladder (Invitrogen), lane 2 is the digest of pGVB-2001-015, and lane 3 is the digest of pGVB-2001-016.

FIG. 6 is an image of a Southern blot showing the restriction digestion of the plasmids containing the dnRhoA gene with a SmaI restriction enzyme, viewed on a 1% agarose gel; lane 1 is a 1 kb ladder (Invitrogen), lane 2 is the digest of pGVB-2001-015, and lane 3 is the digest of pGVB-2001-016.

FIG. 7 is a graph showing the ROCK activity in HTM cells after treatment with three different ROCK activators: Sphingosine-1 phosphate (S1P) at 1 μM, Oleoyl L-lysophosphatidic acid (LPA) at 10 μM, and Dimethyloxalylglycine (DMOG) at 0.5 mM); and one ROCK inhibitor, Y-27632 at 50 μM.

FIG. 8 is a graph showing the ROCK activity level in non-infected HTM cells, HTM cells infected with wild-type scAAV2 capsid containing the dnRhoA gene, or HTM cells infected with Y3 mutated scAAV2 capsid containing the dnRhoA gene; three independent donors of HTM cells were tested and three technical replicates of AAV infection were analyzed; data is presented as the mean±SEM and a one-way ANOVA followed by a Dunnett post-test was performed to compare between groups; * indicates a p-value <0.033 and ** indicates a p-value <0.002.

FIG. 9 is a graph showing the IOP as measured by an Icare TonoLab tonometer before and after Ad5.BMP2 injection; the average IOP value of 6 eyes are shown in each bar; statistical analysis was performed with one-way ANOVA.

FIG. 10 is a graph showing the IOP as measured by an Icare TonoLab tonometer after scAAV2.Y3.CBh.dnRhoA injections.

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety. In case of a conflict in terminology, the present specification is controlling.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three-letter code, both in accordance with 37 C.F.R. § 1.822 and established usage.

Except as otherwise indicated, standard methods known to those skilled in the art may be used for production of recombinant and synthetic polypeptides, antibodies or antigen-binding fragments thereof, manipulation of nucleic acid sequences, production of transformed cells, the construction of recombinant AAV (rAAV) constructs, modified capsid proteins, packaging vectors expressing the AAV rep and/or cap sequences, and transiently and stably transfected packaging cells. Such techniques are known to those skilled in the art. See, e.g., SAMBROOK et al., MOLECULAR CLONING: A LABORATORY MANUAL 4th Ed. (Cold Spring Harbor, N Y, 2012); F. M. AUSUBEL et al. CURRENT PROTOCOLS IN MOLECULAR BIOLOGY (Green Publishing Associates, Inc. and John Wiley & Sons, Inc., New York).

All publications, patent applications, patents, nucleotide sequences, amino acid sequences and other references mentioned herein are incorporated by reference in their entirety.

### Definitions

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of as used herein should not be interpreted as equivalent to “comprising.”

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence or between the two ends (e.g., between domains) such that the function of the polynucleotide or polypeptide is not materially altered. The total of ten or less additional nucleotides or amino acids includes the total number of additional nucleotides or amino acids added together. The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in biological activity of at least about 50% or more as compared to the activity of a polypeptide consisting of the recited sequence.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

A “therapeutically effective” or “treatment effective” amount as used herein is an amount that provides some improvement or benefit to the subject. Alternatively stated, a “therapeutically effective” or “treatment effective” amount is an amount that will provide some alleviation, mitigation, or decrease in at least one clinical symptom in the subject (e.g., reduce the TOP and/or reduce ocular degeneration). Those skilled in the art will appreciate that the therapeutic effects need not be complete or curative, as long as some benefit is provided to the subject.

By the term “treat,” “treating,” or “treatment of” (or grammatically equivalent terms) is meant to reduce or to at least partially improve or ameliorate the severity of the subject's condition and/or to alleviate, mitigate or decrease in at least one clinical symptom and/or to delay the progression of the condition.

As used herein, the term “prevent,” “prevents,” or “prevention” (and grammatical equivalents thereof) means to delay or inhibit the onset of a disease. The terms are not meant to require complete abolition of disease, and encompass any type of prophylactic treatment to reduce the incidence of the condition or delay the onset of the condition.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

As used herein, the terms “protein” and “polypeptide” are used interchangeably and encompass both peptides and proteins, unless indicated otherwise.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be a sense strand or an antisense strand.

As used herein, the term “gene” refers to a nucleic acid molecule capable of being used to produce mRNA, antisense RNA, miRNA, and the like. Genes may or may not be capable of being used to produce a functional protein. Genes can include both coding and non-coding regions (e.g., introns, regulatory elements, promoters, enhancers, termination sequences and 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The terms “5′ portion” and “3′ portion” are relative terms to define a spatial relationship between two or more elements. Thus, for example, a “3′ portion” of a polynucleotide indicates a segment of the polynucleotide that is downstream of another segment. The term “3′ portion” is not intended to indicate that the segment is necessarily at the 3′ end of the polynucleotide, or even that it is necessarily in the 3′ half of the polynucleotide, although it may be. Likewise, a “5′ portion” of a polynucleotide indicates a segment of the polynucleotide that is upstream of another segment. The term “5′ portion” is not intended to indicate that the segment is necessarily at the 5′ end of the polynucleotide, or even that it is necessarily in the 5′ half of the polynucleotide, although it may be.

As used herein with respect to nucleic acids, the term “operably linked” refers to a functional linkage between two or more nucleic acids. For example, a promoter sequence may be described as being “operably linked” to a heterologous nucleic acid sequence because the promoter sequences initiates and/or mediates transcription of the heterologous nucleic acid sequence. In some embodiments, the operably linked nucleic acid sequences are contiguous and/or are in the same reading frame.

The term “open reading frame (ORF),” as used herein, refers to the portion of a polynucleotide (e.g., a gene) that encodes a polypeptide, and is inclusive of the initiation start site (i.e., Kozak sequence) that initiates transcription of the polypeptide. The term “coding region” may be used interchangeably with open reading frame.

The term “optimized” or “optimized for expression”, as used herein, refer to a viral particle that has been optimized to increase expression of the gene in the viral vector. In some embodiments, the viral particle is optimized to increase gene expression in an organism (e.g., an animal such as a human, an animal, a plant, a fungus, an archaeon, or a bacterium) and/or optimized for gene expression in a tissue type of said organism (e.g., ocular tissue, brain tissue, muscle tissue, etc.). In some embodiments, the viral particle is optimized by codon optimization of gene coding sequence in the viral vector. In some embodiments, the viral particle is optimized by the use of a specific viral serotype (e.g., AAV2 or AAV5). In some embodiments, the viral particle is optimized by mutating the amino acid sequence of the viral capsid protein. In some embodiments, the optimized viral capsid protein comprises a Y3 mutation (e.g., a Y444F, Y500F, and Y730F mutation). In some embodiments, the optimized viral particle expresses the gene from the viral vector by about 5% to about 1000% (e.g., about 5%, 10%, 15%, 20%, 25%, 30%, 40%, 50%, 60%, 80%, 90%, 100%, 125%, 150%, 175%, 200%, 350%, 400%, 450%, 500%, 600%, 700%, 800%, 900%, or about 1000%) increased expression in the organism and/or tissue type when compared to the reference particle but which have not been optimized.

The term “codon-optimized”, as used herein, refers to a gene coding sequence or fragment of a gene coding sequence that has been optimized to increase expression by substituting one or more codons normally present in a coding sequence (for example, in a wildtype sequence, including, e.g., a coding sequence for a RhoA protein) with a codon for the same (synonymous) amino acid. In this manner, the protein or protein fragment encoded by the gene or gene fragment is identical, but the underlying nucleobase sequence of the gene or gene fragment, or corresponding mRNA, is different. In some embodiments, the optimization substitutes one or more rare codons (that is, codons for tRNA that occur relatively infrequently in cells from a particular species) with synonymous codons that occur more frequently to improve the efficiency of translation. For example, in human codon-optimization one or more codons in a coding sequence are replaced by codons that occur more frequently in human cells for the same amino acid. Codon optimization can also increase gene or gene fragment expression through other mechanisms that can improve efficiency of transcription and/or translation. Strategies include, without limitation, increasing total GC content (that is, the percent of guanines and cytosines in the entire coding sequence), decreasing CpG content (that is, the number of CG or GC dinucleotides in the coding sequence), removing cryptic splice donor or acceptor sites, and/or adding or removing ribosomal entry and/or initiation sites, such as Kozak sequences. Desirably, a codon-optimized gene or gene fragment exhibits improved protein expression, for example, the protein or protein fragment encoded thereby is expressed at a detectably greater level in a cell compared with the level of expression of the protein or protein fragment provided by the wildtype gene or gene fragment in an otherwise similar cell. Codon-optimization also provides the ability to distinguish a codon-optimized gene and/or corresponding mRNA from an endogenous gene and/or corresponding mRNA in vitro or in vivo.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

As used herein, the term “modified,” as applied to a polynucleotide or polypeptide sequence, refers to a sequence that differs from a wildtype sequence due to one or more deletions, additions, substitutions, or any combination thereof.

As used herein, by “isolate” (or grammatical equivalents) a virus vector, it is meant that the virus vector is at least partially separated from at least some of the other components in the starting material.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “substantially identical” or “corresponding to” means that two nucleic acid sequences have at least 60%, 70%, 80% or 90% sequence identity. In some embodiments, the two nucleic acid sequences can have at least 85%, 90%, 95%, 96%, 97%, 98%, 99% or 100% of sequence identity.

An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., Burlington, Mass.). Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo, H., and Lipton, D., (Applied Math 48:1073 (1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

A “vector” refers to a compound used as a vehicle to carry foreign genetic material into another cell, where it can be replicated and/or expressed. A cloning vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene/ORF. Insertion of a vector into the target cell is referred to transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

The term “promoter” as used herein refers to a polynucleotide sequence to which a polymerase (DNA or RNA) or associated transcription factors bind to begin transcription. In some embodiments, the promoter may be a promoter sequence from the genome of a virus (e.g., CMV, AAV, etc.), prokaryotic (e.g., Escherichia coli), or eukaryotic organism (e.g., human, chicken, mouse, yeast, etc.). In some embodiments, the eukaryotic promoter has low immunogenicity, e.g., the viral vector comprising the eukaryotic promoter has an anti-drug antibody (ADA) response rate of about 1% to about 30% (e.g., about 1%, 2%, 3%, 4%, 5%, 10%, 15%, 20%, 25%, or about 30%). In some embodiments, the eukaryotic promoter has stable expression in the subject, e.g., expression from the viral vector comprising the eukaryotic promoter has a variance of expression of about 1% to about 40% (e.g., about 1%, 2%, 3%, 4%, 5%, 10%, 15%, 20%, 25%, 30%, 35%, or about 40%) from a baseline expression level after administration to said subject. In some embodiments, the promoter may be a short promoter, e.g., has a sequence length of about 100 nucleotides to about 2,000 nucleotides (e.g., about 100, 200, 300, 400, 500, 600, 700, 800, 900, 1000, 1100, or about 1200 nucleotides in length). In some embodiments, the short promoter is about 500 to about 1000 nucleotides in length (e.g., about 500, 600, 700, 800, 900, or about 1000 nucleotides). In some embodiments, the promoter is a CMV5, CBA, or an EF1α promoter (e.g., a truncated EF1α promoter). In some embodiments, the EF1α promoter is about 1000 to about 1400 nucleotides in length (e.g., about 1000, 1100, 1200, 1300, or about 1400 nucleotides). In some embodiments, the promoter may be a hybrid promoter, e.g., a promoter where two promoter sequences from one or more different organism are operably linked to one another. In some embodiments, a hybrid promoter is a CBh promoter (e.g., a CMV and chicken beta-actin hybrid promoter). In some embodiments, the CBA and/or CBh promoter is about 400 to about 1000 nucleotides in length (e.g., about 400, 500, 600, 700, 800, 900, or about 1000 nucleotides).

A “subject” of the invention may include any animal in need thereof. In some embodiments, a subject may be, for example, a mammal, a reptile, a bird, an amphibian, or a fish. A mammalian subject may include, but is not limited to, a laboratory animal (e.g., a rat, mouse, guinea pig, rabbit, primate, etc.), a farm or commercial animal (e.g., cattle, pig, horse, goat, donkey, sheep, etc.), or a domestic animal (e.g., cat, dog, ferret, gerbil, hamster, etc.). In some embodiments, a mammalian subject may be a primate, or a non-human primate (e.g., a chimpanzee, baboon, macaque (e.g., rhesus macaque, crab-eating macaque, stump-tailed macaque, pig-tailed macaque), monkey (e.g., squirrel monkey, owl monkey, etc.), marmoset, gorilla, etc.). In some embodiments, a mammalian subject may be a human.

A “subject in need” of the methods of the invention can be any subject known or suspected of having increased risk of developing an ocular disease (e.g., glaucoma, AMD (e.g., dry AMD or wet AMD), diabetic retinopathy, and/or retinal holes), and/or ocular hypertension (increased IOP).

### Compositions and Formulations

Provided herein are recombinant scAAV vectors and methods for the treatment of an ocular disease (e.g., glaucoma), and/or ocular hypertension using the same. Accordingly, the disclosure provides recombinant scAAV particles, compositions comprising recombinant scAAV particles, and methods for the treatment of an ocular disease (e.g., glaucoma), and/or ocular hypertension. In some embodiments, the disclosed viral vectors may be used in conjunction with any AAV capsid, with or without mutations, in the manufacture of an AAV particle for the treatment of an ocular disease (e.g., glaucoma), and/or ocular hypertension, or any combination thereof. Further provided herein are pharmaceutical formulations and doses of viral injectate that can be used in intraocular injections. Another aspect of the disclosure provided herein is injection routes that are commonly used in treating an ocular disease (e.g., glaucoma), and/or ocular hypertension, or any combination thereof. Other aspects of the disclosure related to the therapeutic effects of the viral vectors in glaucoma patients.

The current inventors have been optimizing the delivery vector, its cargo, and the potential mechanism to regulate the expression of a cargo gene. For the delivery vehicle, the inventors sought a long TM duration, low immunogenicity, and serotype-selected and mutated capsid virus with increased gene transfer efficiency. For the cargo, an efficient dominant negative RhoA mutation was identified to reduce IOP in glaucoma animal models. For the promoters, a hybrid chicken beta-actin (CBh) promoter was selected to drive transgene expression. Taken together, a viral vector of the present invention may contain a RhoA gene comprising a dominant negative mutation (e.g., SEQ ID NO: 4) and a ubiquitous promoter such as CBh (SEQ ID NO: 3). The viral vector (SEQ ID NO: 7) was manufactured and characterized in vitro and consequently tested in rat glaucoma models for its efficacy.

In some embodiments, the AAV particles can have altered VP1 capsid proteins, altered VP2 capsid proteins, altered VP3 capsid proteins, or any combination thereof. In some embodiments of the aspects and embodiments described above, the AAV viral particle comprises AAV1, AAV2, AAV3, AAV4, AAV5, AAV6, AAV7, AAV8, AAVrh8, AAVrh8R, AAV9, AAV10, AAVrh10, AAV11, AAV12, mutant capsid containing tyrosine, mutant capsid with heparin-binding motif, capsid AAV2R471A, capsid AAV2/2, and/or capsid AAV DJ. In some embodiments, the AAV viral particle comprises an AAV capsid containing an amino acid substitution at one or more positions Y444F, Y500F, and/or Y730F, numbered according to VP1 of AAV2 (SEQ ID NO:5). In some embodiments, the vector contains inverted terminal repeats (ITR) of serotypes AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV12, AAV2R471A, bovine AAV and/or mouse AAV. In some embodiments, the vector comprises a mutated AAV ITR, which leads to the formation of self-complemented AAV. In some embodiments, the AAV viral particle comprises one or more ITRs and a capsid derived from the same AAV serotype. In other embodiments, the AAV viral particle contains one or more ITRs derived from an AAV serotype other than that of the recombinant scAAV viral particle capsid. In some embodiments, the recombinant scAAV viral particle comprises an AAV2 capsid, and wherein the vector comprises an AAV2 ITR.

In some embodiments, various formulations may be used to facilitate viral transduction in ocular tissue. For example, for administration of an injectable aqueous solution of recombinant scAAV particles, the solution may be suitably buffered, if necessary, and the liquid diluent first rendered isotonic with sufficient saline or glucose. Accordingly, in some embodiments, the viral injectate may comprise a pharmaceutically acceptable carrier. The term “carrier” refers to a diluent, adjuvant, excipient, or vehicle with which the recombinant scAAV particle is administered. By “pharmaceutically acceptable” it is meant a material that is not toxic or otherwise undesirable, i.e., the material may be administered to a subject without causing any undesirable biological effects. Such pharmaceutical carriers can be sterile liquids (e.g., water, oils, saline solutions, aqueous dextrose and/or glycerol solutions), suspending agents, preserving agents (e.g., methyl-, ethyl-, and/or propyl-hydroxy-benzoates), and pH adjusting agents (such as inorganic acids, organic acids, and/or bases). In some embodiments, carriers include buffered saline solutions (e.g., phosphate buffered saline, HEPES-buffered saline, etc.). In some embodiments, United States Pharmacopeia (USP) grade carriers and excipients may be used for the delivery of recombinant scAAV particles to human subjects. Such compositions may further optionally comprise a liposome, a lipid, a lipid complex, a microsphere, a microparticle, a nanosphere, and/or a nanoparticle, or may be otherwise formulated for administration to the cells, tissues, organs, or body of a subject in need thereof.

In some embodiments, a composition comprising any one of the recombinant scAAV particles disclosed herein comprises a Balanced Salt Solution (BSS) supplemented with about 0.001% to about 0.035% Tween 20 (polysorbate 20) (e.g., about 0.001%, 0.007%, 0.014%, 0.021%, 0.028%, or about 0.035% or any range therein). In some embodiments, a composition comprising any one of the recombinant scAAV particles disclosed herein comprises from about 25 mM to about 500 mM sodium citrate (e.g., about 25, 50, 75, 100, 125, 150, 175, 200, 225, 250, 300, 350, 400, 450, or about 500 mM or any range therein), from about 1 mM to about 50 mM Tris (e.g., about 1, 2, 3, 4, 5, 6, 7, 8, 9, 10, 15, 20, 25, 30, 35, 40, 45, or about 50 mM or any range therein), pH 8.0, supplemented with about 0.0001% to about 0.005% Pluronic F-68 (e.g., about 0.0001%, 0.0002%, 0.0003%, 0.0004%, 0.0005%, 0.0006%, 0.0007%, 0.0008%, 0.0009%, 0.001%, 0.002%, 0.003%, 0.004%, or about 0.005% or any range therein).

### Methods of Use

The methods of the present invention find use in both veterinary and medical applications.

Dosages of the inventive AAV particles depend upon the mode of administration, the disease or condition to be treated, the individual subject's condition, the viral vector, and the gene to be delivered, and can be determined in a routine manner. Exemplary doses for achieving therapeutic effects are virus titers of at least about 10⁵, 10⁶, 10⁷, 10⁸, 10⁹, 10¹⁰, 10¹¹, 10¹², 10¹³, 10¹⁴, 10¹⁵ transducing units or more, preferably about 10⁸-10¹³ transducing units, yet more preferably 10¹¹ transducing units.

The term “administering” or “administration” of a composition of the present invention to a subject includes any route of introducing or delivering to a subject an agent to perform its intended function (e.g., for reducing TOP or treating glaucoma in a subject). In some embodiments, the recombinant scAAV viral vectors may be administered via intraocular injection. The term “intraocular injection”, as used herein, refers to any convenient injection route that can result in the delivery of the recombinant scAAV vector to the ocular tissues (e.g., any part of the eye). In some embodiments, the intraocular injection is an injection into the anterior portion (e.g., anterior chamber) of the eye, e.g., by intracameral injection. In some embodiments, the intraocular injection is an injection into the posterior portion (e.g., posterior chamber) of the eye, e.g., into the vitreous of the eye, e.g., by intravitreal injection. In some embodiments, the intraocular injection is an injection into the cornea of the eye, e.g., by intrastromal corneal injection. In some embodiments, the intraocular injection is an injection under the sensory retina of the eye, e.g., by subretinal injection. In some embodiments, the intraocular injection is an injection into the subretinal space of the eye (e.g., the suprachoroidal space), e.g., by suprachoroidal injection. Other convenient routes of administration include, without limitation, intravenous, intra-arterial, periocular, subconjunctival and sub-tenons injections, topical administration (e.g., topical administration to the eye), and intranasal administration.

The present viral vectors may be effective in treating or preventing neurological dysfunction. In some embodiments, the viral vectors described herein may provide IOP-independent or IOP-dependent neuroprotective effects on retinal cells, such as RGCs. In some embodiments, the viral vectors described herein prevent progression of glaucoma by reducing IOP and/or by providing direct neuroprotection. In certain embodiments, the viral vectors may prevent progression of glaucoma by both providing direct neuroprotection and reducing intraocular pressure. This therapy offers an unexpected dual benefit in the treatment of glaucoma. In some embodiments, the viral vectors described herein may prevent progression of glaucoma without reducing IOP. Thus, this method of treatment may provide an unexpected benefit in an TOP-independent neuroprotective effect.

In some embodiments, the viral vectors described herein may accelerate the wound healing of cornea after injury. Thus, this method of treatment provides an unexpected benefit in cornea protection.

Having described the present invention, the same will be explained in greater detail in the following examples, which are included herein for illustration purposes only, and which are not intended to be limiting to the invention.

Promoter CMV5
SEQ ID NO: 1
CGCGTTACATAACTTACGGTAAATGGCCCGCCTGGCTGACCGCCC
AACGACCCCCGCCCATTGACGTCAATAATGACGTATGTTCCCATA
GTAACGCCAATAGGGACTTTCCATTGACGTCAATGGGTGGAGTAT
TTACGGTAAACTGCCCACTTGGCAGTACATCAAGTGTATCATATG
CCAAGTCCGCCCCCTATTGACGTCAATGACGGTAAATGGCCCGCC
TGGCATTATGCCCAGTACATGACCTTACGGGACTTTCCTACTTGG
CAGTACATCTACGTATTAGTCATCGCTATTACCATGGTGATGCGG
TTTTGGCAGTACACCAATGGGCGTGGATAGCGGTTTGACTCACGG
GGATTTCCAAGTCTCCACCCCATTGACGTCAATGGGAGTTTGTTT
TGGCACCAAAATCAACGGGACTTTCCAAAATGTCGTAATAACCCC
GCCCCGTTGACGCAAATGGGCGGTAGGCGTGTACGGTGGGAGGTC
TATATAAGCAGAGCTCGTTTAGTGAACCGTCAGATCCTCACTCTC
TTCCGCATCGCTGTCTGCGAGGGCCAGCTGTTGGGCTCGCGGTTG
AGGACAAACTCTTCGCGGTCTTTCCAGTACTCTTGGATCGGAAAC
CCGTCGGCCTCCGAACGGTACTCCGCCACCGAGGGACCTGAGCGA
GTCCGCATCGACCGGATCGGAAAACCTCTCGAGAAAGGCGTCTAA
CCAGTCACAGTCGCAAGGTAGGCTGAGCACCGTGGCGGGCGGCAG
CGGGTGGCGGTCGGGGTTGTTTCTGGCGGAGGTGCTGCTGATGAT
GTAATTAAAGTAGGCGGTCTTGAGACGGCGGATGGTCGAGGTGAG
GTGTGGCCGGCTTGAGATCCAGCTGTTGGGGTGAGTACTCCCTCT
CAAAAGCGGGCATGACTTCTGCGCTAAGATTGTCAGTTTCCAAAA
ACGAGGAGGATTTGATATTCACCTGGCCCGATCTGGCCATACACT
TGAGTGACAATGACATCCACTTTGCCTTTCTCTCCACAGGTGTCC
ACTCCCAGGTCCAA
Promoter truncated EFla
SEQ ID NO: 2
GAACCGGTGCCTAGAGAAGGTGGCGCGGGGTAAACTGGGAAAGTG
ATGTCGTGTACTGGCTCCGCCTTTTTCCCGAGGGTGGGGGAGAAC
CGTATATAAGTGCAGTAGTCGCCGTGAACGTTCTTTTTCGCAACG
GGTTTGCCGCCAGAACACAGGTAAGTGCCGTGTGTGGTTCCCGCG
GGCCTGGCCTCTTTACGGGTTATGGCCCTTGCGTGCCTTGAATTA
CTTCCACGCCCCTGGCTGCAGTACGTGATTCTTGATCCCGAGCTT
CGGGTTGGAAGTGGGTGGGAGAGTTCGAGGCCTTGCGCTTAAGGA
GCCCCTTCGCCTCGTGCTTGAGTTGAGGCCTGGCCTGGGCGCTGG
GGCCGCCGCGTGCGAATCTGGTGGCACCTTCGCGCCTGTCTCGCT
GCTTTCGATAAGTCTCTAGCCATTTAAAATTTTTGATGACCTGCT
GCGACGCTTTTTTTCTGGCAAGATAGTCTTGTAAATGCGGGCCAA
GATCTGCACACTGGTATTTCGGTTTTTGGGGCCGCGGGCGGCGAC
GGGGCCCGTGCGTCCCAGCGCACATGTTCGGCGAGGCGGGGCCTG
CGAGCGCGGCCACCGAGAATCGGACGGGGGTAGTCTCAAGCTGGC
CGGCCTGCTCTGGTGCCTGGCCTCGCGCCGCCGTGTATCGCCCCG
CCCTGGGCGGCAAGGCTGGCCCGGTCGGCACCAGTTGCGTGAGCG
GAAAGATGGCCGCTTCCCGGCCCTGCTGCAGGGAGCTCAAAATGG
AGGACGCGGCGCTCGGGAGAGCGGGCGGGTGAGTCACCCACACAA
AGGAAAAGGGCCTTTCCGTCCTCAGCCGTCGCTTCATGTGACTCC
ACGGAGTACCGGGCGCCGTCCAGGCACCTCGATTAGTTCTCGAGC
TTTTGGAGTACGTCGTCTTTAGGTTGGGGGGAGGGGTTTTATGCG
ATGGAGTTTCCCCACACTGAGTGGGTGGAGACTGAAGTTAGGCCA
GCTTGGCACTTGATGTAATTCTCCTTGGAATTTGCCCTTTTTGAG
TTTGGATCTTGGTTCATTCTCAAGCCTCAGACAGTGGTTCAAAGT
TTTTTTCTTCCATTTCAGGTGTCGTGA
Promoter hybrid chicken beta-actin (CBh)
SEQ ID NO: 3
CGTTACATAACTTACGGTAAATGGCCCGCCTGGCTGACCGCCCAA
CGACCCCCGCCCATTGACGTCAATAGTAACGCCAATAGGGACTTT
CCATTGACGTCAATGGGTGGAGTATTTACGGTAAACTGCCCACTT
GGCAGTACATCAAGTGTATCATATGCCAAGTACGCCCCCTATTGA
CGTCAATGACGGTAAATGGCCCGCCTGGCATTGTGCCCAGTACAT
GACCTTATGGGACTTTCCTACTTGGCAGTACATCTACGTATTAGT
CATCGCTATTACCATGGTCGAGGTGAGCCCCACGTTCTGCTTCAC
TCTCCCCATCTCCCCCCCCTCCCCACCCCCAATTTTGTATTTATT
TATTTTTTAATTATTTTGTGCAGCGATGGGGGCGGGGGGGGGGGG
GGGGCGCGCGCCAGGCGGGGCGGGGCGGGGCGAGGGGCGGGGCGG
GGCGAGGCGGAGAGGTGCGGCGGCAGCCAATCAGAGCGGCGCGCT
CCGAAAGTTTCCTTTTATGGCGAGGCGGCGGCGGCGGCGGCCCTA
TAAAAAGCGAAGCGCGCGGCGGGCG
Amino acid sequence of dominant negative
RhoA (T19N)
SEQ ID NO: 4
MAAIRKKLVIVGDGACGKNCLLIVESKDQFPEVYVPTVFENYVAD
IEVDGKQVELALWDTAGQEDYDRLRPLSYPDTDVILMCFSIDSPD
SLENIPEKWTPEVKHFCPNVPIILVGNKKDLRNDEHTRRELAKMK
QEPVKPEEGRDMANRIGAFGYMECSAKTKDGVREVFEMATRAALQ
ARRGKKKSGCLVL
Amino acid sequence of AAV2 VP1
SEQ ID NO: 5
MAADGYLPDWLEDTLSEGIRQWWKLKPGPPPPKPAERHKDDSRGL
VLPGYKYLGPFNGLDKGEPVNEADAAALEHDKAYDRQLDSGDNPY
LKYNHADAEFQERLKEDTSFGGNLGRAVFQAKKRVLEPLGLVEEP
VKTAPGKKRPVEHSPVEPDSSSGTGKAGQQPARKRLNFGQTGDAD
SVPDPQPLGQPPAAPSGLGTNTMATGSGAPMADNNEGADGVGNSS
GNWHCDSTWMGDRVITTSTRTWALPTYNNHLYKQISSQSGASNDN
HYFGYSTPWGYFDFNRFHCHFSPRDWQRLINNNWGFRPKRLNFKL
FNIQVKEVTQNDGTTTIANNLTSTVQVFTDSEYQLPYVLGSAHQG
CLPPFPADVFMVPQYGYLTLNNGSQAVGRSSFYCLEYFPSQMLRT
GNNFTFSYTFEDVPFHSSYAHSQSLDRLMNPLIDQYLYYLSRTNT
PSGTTTQSRLQFSQAGASDIRDQSRNWLPGPCYRQQRVSKTSADN
NNSEYSWTGATKYHLNGRDSLVNPGPAMASHKDDEEKFFPQSGVL
IFGKQGSEKTNVDIEKVMITDEEEIRTTNPVATEQYGSVSTNLQR
GNRQAATADVNTQGVLPGMVWQDRDVYLQGPIWAKIPHTDGHFHP
SPLMGGFGLKHPPPQILIKNTPVPANPSTTFSAAKFASFITQYST
GQVSVEIEWELQKENSKRWNPEIQYTSNYNKSVNVDFTVDTNGVY
SEPRPIGTRYLTRNL
Amino acid sequence of wild type RhoA
SEQ ID NO: 6
MAAIRKKLVIVGDGACGKTCLLIVESKDQFPEVYVPTVFENYVAD
IEVDGKQVELALWDTAGQEDYDRLRPLSYPDTDVILMCFSIDSPD
SLENIPEKWTPEVKHFCPNVPIILVGNKKDLRNDEHTRRELAKMK
QEPVKPEEGRDMANRIGAFGYMECSAKTKDGVREVFEMATRAALQ
ARRGKKKSGCLVL
Sequence of a scAAV2.CBh.dnRhoA
vector comprising a CBh promoter
and dnRhoA gene
SEQ ID NO: 7
CTGGCGCGCTCGCTCGCTCACTGAGGCCGCCCGGGCAAAGCCCGG
GCGTCGGGCGACCTTTGGTCGCCCGGCCTCAGTGAGCGAGCGAGC
GCGCAGAGAGGGAGTGGCCAACTCCATCACTAGGGGTTCCTTGTA
GTTAATGATTAACCCGCCATGCTACTTATCTACGTAGCCATGCTC
TAGGATCTGAATTCGGTACCCGTTACATAACTTACGGTAAATGGC
CCGCCTGGCTGACCGCCCAACGACCCCCGCCCATTGACGTCAATA
ATGACGTATGTTCCCATAGTAACGCCAATAGGGACTTTCCATTGA
CGTCAATGGGTGGAGTATTTACGGTAAACTGCCCACTTGGCAGTA
CATCAAGTGTATCATATGCCAAGTACGCCCCCTATTGACGTCAAT
GACGGTAAATGGCCCGCCTGGCATTATGCCCAGTACATGACCTTA
TGGGACTTTCCTACTTGGCAGTACATCTACGTATTAGTCATCGCT
ATTACCATGGTCGAGGTGAGCCCCACGTTCTGCTTCACTCTCCCC
ATCTCCCCCCCCTCCCCACCCCCAATTTTGTATTTATTTATTTTT
TAATTATTTTGTGCAGCGATGGGGGCGGGGGGGGGGGGGGGGGGC
GCGCCAGGCGGGGCGGGGCGGGGCGAGGGGCGGGGCGGGGCGAGG
CGGAGAGGTGCGGCGGCAGCCAATCAGAGCGGCGCGCTCCGAAAG
TTTCCTTTTATGGCGAGGCGGCGGCGGCGGCGGCCCTATAAAAAG
CGAAGCGCGCGGCGGGCGGGAGTCGCTGCGACGCTGCCTTCGCCC
CGTGCCCCGCTCCGCCGCCGCCTCGCGCCGCCCGCCCCGGCTCTG
ACTGACCGCGTTACTCCCACAGGTGAGCGGGCGGGACGGCCCTTC
TCCTCCGGGCTGTAATTAGCTGAGCAAGAGGTAAGGGTTTAAGGG
ATGGTTGGTTGGTGGGGTATTAATGTTTAATTACCTGGAGCACCT
GCCTGAAATCACTTTTTTTCAGGTTGGGAGCAATGGCTGCCATCC
GGAAGAAACTGGTGATTGTTGGTGATGGAGCCTGTGGAAAGAACT
GCTTGCTCATAGTCTTCAGCAAGGACCAGTTCCCAGAGGTGTATG
TGCCCACAGTGTTTGAGAACTATGTGGCAGATATCGAGGTGGATG
GAAAGCAGGTAGAGTTGGCTTTGTGGGACACAGCTGGGCAGGAAG
ATTATGATCGCCTGAGGCCCCTCTCCTACCCAGATACCGATGTTA
TACTGATGTGTTTTTCCATCGACAGCCCTGATAGTTTAGAAAACA
TCCCAGAAAAGTGGACCCCAGAAGTCAAGCATTTCTGTCCCAACG
TGCCCATCATCCTGGTTGGGAATAAGAAGGATCTTCGGAATGATG
AGCACACAAGGCGGGAGCTAGCCAAGATGAAGCAGGAGCCGGTGA
AACCTGAAGAAGGCAGAGATATGGCAAACAGGATTGGCGCTTTTG
GGTACATGGAGTGTTCAGCAAAGACCAAAGATGGAGTGAGAGAGG
TTTTTGAAATGGCTACGAGAGCTGCTCTGCAAGCTAGACGTGGGA
AGAAAAAATCTGGTTGCCTTGTCTTGTGAGCGGCCGCGGGGATCC
AGACATGATAAGATACATTGATGAGTTTGGACAAACCACAACTAG
AATGCAGTGAAAAAAATGCTTTATTTGTGAAATTTGTGATGCTAT
TGCTTTATTTGTAACCATTATAAGCTGCAATAAACAAGTTAACAA
CAACAATTGCATTCATTTTATGTTTCAGGTTCAGGGGGAGGTGTG
GGAGGTTTTTTAGTCGACTAGAGCTCGCTGATCAGCCTCGACTGT
GCCTTCTAGTTGCCAGCCATCTGTTGTTTGCCCCTCCCCCGTGCC
TTCCTTGACCCTGGAAGGTGCCACTCCCACTGTCCTTTCCTAATA
AAATGAGGAAATTGCATCGCATTGTCTGAGTAGGTGTCATTCTAT
TCTGGGGGGTGGGGTGGGGCAGGACAGCAAGGGGGAGGATTGGGA
AGACAATAGCAGGAACCCCACTCCCTCTCTGCGCGCTCGCTCGCT
CACTGAGGCCGGGCGACCAAAGGTCGCCCGACGCCCGGGCTTTGC
CCGGGCGGCCTCAGTGAGCGAGCGAGCGCGCAGCTGCTGCATTAA
TGAATCGGCCAACGCGCGGGGAGAGGCGGTTTGCGTATTGGGCGC
TCTTCCGCTTCCTCGCTCACTGACTCGCTGCGCTCGGTCGTTCGG
CTGCGGCGAGCGGTATCAGCTCACTCAAAGGCGGTAATACGGTTA
TCCACAGAATCAGGGGATAACGCAGGAAAGAACATGTGAGCAAAA
GGCCAGCAAAAGGCCAGGAACCGTAAAAAGGCCGCGTTGCTGGCG
TTTTTCCATAGGCTCCGCCCCCCTGACGAGCATCACAAAAATCGA
CGCTCAAGTCAGAGGTGGCGAAACCCGACAGGACTATAAAGATAC
CAGGCGTTTCCCCCTGGAAGCTCCCTCGTGCGCTCTCCTGTTCCG
ACCCTGCCGCTTACCGGATACCTGTCCGCCTTTCTCCCTTCGGGA
AGCGTGGCGCTTTCTCATAGCTCACGCTGTAGGTATCTCAGTTCG
GTGTAGGTCGTTCGCTCCAAGCTGGGCTGTGTGCACGAACCCCCC
GTTCAGCCCGACCGCTGCGCCTTATCCGGTAACTATCGTCTTGAG
TCCAACCCGGTAAGACACGACTTATCGCCACTGGCAGCAGCCACT
GGTAACAGGATTAGCAGAGCGAGGTATGTAGGCGGTGCTACAGAG
TTCTTGAAGTGGTGGCCTAACTACGGCTACACTAGAAGGACAGTA
TTTGGTATCTGCGCTCTGCTGAAGCCAGTTACCTTCGGAAAAAGA
GTTGGTAGCTCTTGATCCGGCAAACAAACCACCGCTGGTAGCGGT
GGTTTTTTTGTTTGCAAGCAGCAGATTACGCGCAGAAAAAAAGGA
TCTCAAGAAGATCCTTTGATCTTTTCTACGGGGTCTGACGCTCAG
TGGAACGAAAACTCACGTTAAGGGATTTTGGTCATGAGATTATCA
AAAAGGATCTTCACCTAGATCCTTTTAAATTAAAAATGAAGTTTT
AAATCAATCTAAAGTATATATGAGTAAACTTGGTCTGACAGTCAG
AAGAACTCGTCAAGAAGGCGATAGAAGGCGATGCGCTGCGAATCG
GGAGCGGCGATACCGTAAAGCACGAGGAAGCGGTCAGCCCATTCG
CCGCCAAGCTCTTCAGCAATATCACGGGTAGCCAACGCTATGTCC
TGATAGCGGTCCGCCACACCCAGCCGGCCACAGTCGATGAATCCA
GAAAAGCGGCCATTTTCCACCATGATATTCGGCAAGCAGGCATCG
CCATGGGTCACGACGAGATCCTCGCCGTCGGGCATGCTCGCCTTG
AGCCTGGCGAACAGTTCGGCTGGCGCGAGCCCCTGATGCTCTTCG
TCCAGATCATCCTGATCGACAAGACCGGCTTCCATCCGAGTACGT
GCTCGCTCGATGCGATGTTTCGCTTGGTGGTCGAATGGGCAGGTA
GCCGGATCAAGCGTATGCAGCCGCCGCATTGCATCAGCCATGATG
GATACTTTCTCGGCAGGAGCAAGGTGAGATGACAGGAGATCCTGC
CCCGGCACTTCGCCCAATAGCAGCCAGTCCCTTCCCGCTTCAGTG
ACAACGTCGAGCACAGCTGCGCAAGGAACGCCCGTCGTGGCCAGC
CACGATAGCCGCGCTGCCTCGTCTTGCAGTTCATTCAGGGCACCG
GACAGGTCGGTCTTGACAAAAAGAACCGGGCGCCCCTGCGCTGAC
AGCCGGAACACGGCGGCATCAGAGCAGCCGATTGTCTGTTGTGCC
CAGTCATAGCCGAATAGCCTCTCCACCCAAGCGGCCGGAGAACCT
GCGTGCAATCCATCTTGTTCAATCATACTCTTCCTTTTTCAATAT
TATTGAAGCATTTATCAGGGTTATTGTCTCATGAGCGGATACATA
TTTGAATGTATTTAGAAAAATAAACAAATAGGGGTTCCGCGCACA
TTTCCCCGAAAAGTGCCACCTGACGTCTAAGAAACCATTATTATC
ATGACATTAACCTATAAAAATAGGCGTATCACGAGGCCCTTTCGT
CTCGCGCGTTTCGGTGATGACGGTGAAAACCTCTGACACATGCAG
CTCCCGGAGACGGTCACAGCTTGTCTGTAAGCGGATGCCGGGAGC
AGACAAGCCCGTCAGGGCGCGTCAGCGGGTGTTGGCGGGTGTCGG
GGCTGGCTTAACTATGCGGCATCAGAGCAGATTGTACTGAGAGTG
CACCATATGCGGTGTGAAATACCGCACAGATGCGTAAGGAGAAAA
TACCGCATCAGGAATTCCAACATCCAATAAATCATACAGGCAAGG
CAAAGAATTAGCAAAATTAAGCAATAAAGCCTCAGAGCATAAAGC
TAAATCGGTTGTACCAAAAACATTATGACCCTGTAATACTTTTGC
GGGAGAAGCCTTTATTTCAACGCAAGGATAAAAATTTTTAGAACC
CTCATATATTTTAAATGCAATGCCTGAGTAATGTGTAGGTAAAGA
TTCAAACGGGTGAGAAAGGCCGGAGACAGTCAAATCACCATCAAT
ATGATATTCAACCGTTCTAGCTGATAAATTCATGCCGGAGAGGGT
AGCTATTTTTGAGAGGTCTCTACAAAGGCTATCAGGTCATTGCCT
GAGAGTCTGGAGCAAACAAGAGAATCGATGAACGGTAATCGTAAA
ACTAGCATGTCAATCATATGTACCCCGGTTGATAATCAGAAAAGC
CCCAAAAACAGGAAGATTGTATAAGCAAATATTTAAATTGTAAAC
GTTAATATTTTGTTAAAATTCGCGTTAAATTTTTGTTAAATCAGC
TCATTTTTTAACCAATAGGCCGAAATCGGCAAAATCCCTTATAAA
TCAAAAGAATAGACCGAGATAGGGTTGAGTGTTGTTCCAGTTTGG
AACAAGAGTCCACTATTAAAGAACGTGGACTCCAACGTCAAAGGG
CGAAAAACCGTCTATCAGGGCGATGGCCCACTACGTGAACCATCA
CCCTAATCAAGTTTTTTGGGGTCGAGGTGCCGTAAAGCACTAAAT
CGGAACCCTAAAGGGAGCCCCCGATTTAGAGCTTGACGGGGAAAG
CCGGCGAACGTGGCGAGAAAGGAAGGGAAGAAAGCGAAAGGAGCG
GGCGCTAGGGCGCTGGCAAGTGTAGCGGTCACGCTGCGCGTAACC
ACCACACCCGCCGCGCTTAATGCGCCGCTACAGGGCGCGTACTAT
GGTTGCTTTGACGAGCACGTATAACGTGCTTTCCTCGTTAGAATC
AGAGCGGGAGCTAAACAGGAGGCCGATTAAAGGGATTTTAGACAG
GAACGGTACGCCAGAATCCTGAGAAGTGTTTTTATAATCAGTGAG
GCCACCGAGTAAAAGAGTCTGTCCATCACGCAAATTAACCGTTGT
CGCAATACTTCTTTGATTAGTAATAACATCACTTGCCTGAGTAGA
AGAACTCAAACTATCGGCCTTGCTGGTAATATCCAGAACAATATT
ACCGCCAGCCATTGCAACAGGAAAAACGCTCATGGAAATACCTAC
ATTTTGACGCTCAATCGTCTGGAATTCCATTCGCCATTCAGGCTG
CGCAACTGTTGGGAAGGGCGATCGGTGCGGGCCTCTTCGCTATTA
CGCCAG

### EXAMPLES

### Example 1: Comparison of Promoters to Drive GFP Expression in HTM Cells

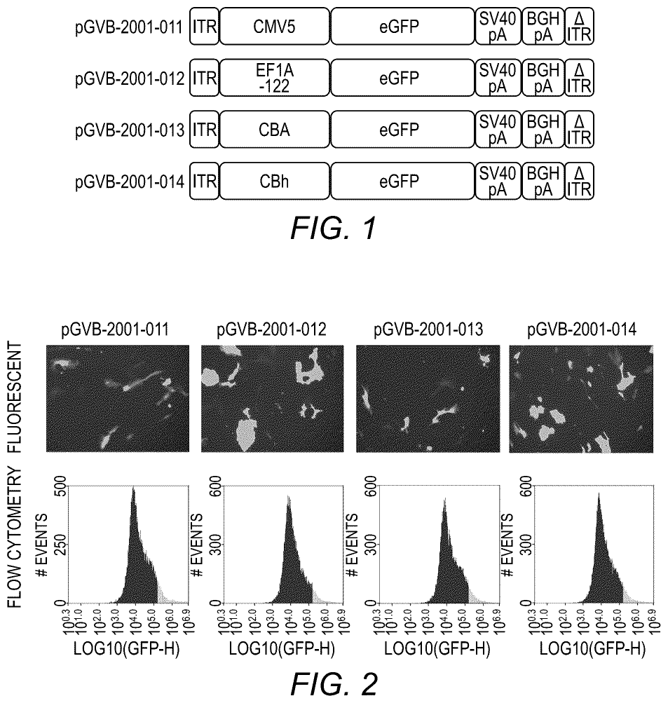

To identify a suitable promoter in HTM cells, the efficiency of several promoters was compared in driving green fluorescent protein (GFP) expression. Four commonly used promoters were selected to construct the plasmids (FIG. 1): CMV5 (SEQ ID NO:1), EF1α (SEQ ID NO:2), and CBh (SEQ ID NO:3). The first step was to test the expression of the new plasmids in HTM cells by Lipofectamine (ThermoFisher) transfection. HTM cells (pooled from two individual donors) were seeded in 24-well plates the day before transfection to reach 70-80% confluency. Cells were transfected with 0.5 μg of plasmids to check the promoter efficiency using Lipofectamine 3000 transfection reagent (ThermoFisher). Cells were grown for 72 hours before being visualized by a fluorescent microscope. Later, cells were examined by flow cytometry to quantify the ratio of GFP positive cells. Overall, the level of GFP expression in HTM cells as recorded by fluorescent microscope and flow cytometry was similar between groups of different promoters (FIG. 2 & FIG. 4).

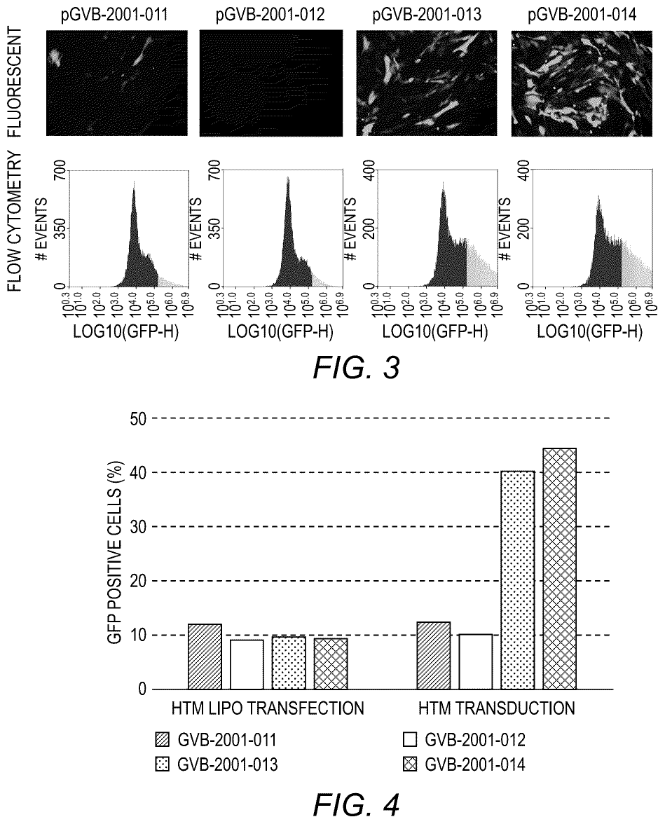

The promoter efficiency was further tested in the form of AAV viral transduction in HTM cells. AAV particles were packaged with plasmids used in previous transfection experiments. Since the plasmids all contain delta-ITR, viral DNA was self-complementary. AAV2 with Y3 mutation (Y444F, Y500F, and Y730F) was selected as the capsid. Then, HTM cells were seeded in 24-well plates. Cells were infected with 10,000 MOI. Cells were grown for 3 days before they were examined. The ratio of GFP positive cells as recorded by flow cytometry showed different results between groups with different promoters. The cells infected with of the vector comprising the CBh promoter were much higher in percentage of GFP positive cells than the groups of cells infected with vectors comprising CMV5 and EF1α promoters (FIG. 3 & FIG. 4).

### Example 2: Construction of scAAV2.dnRhoA

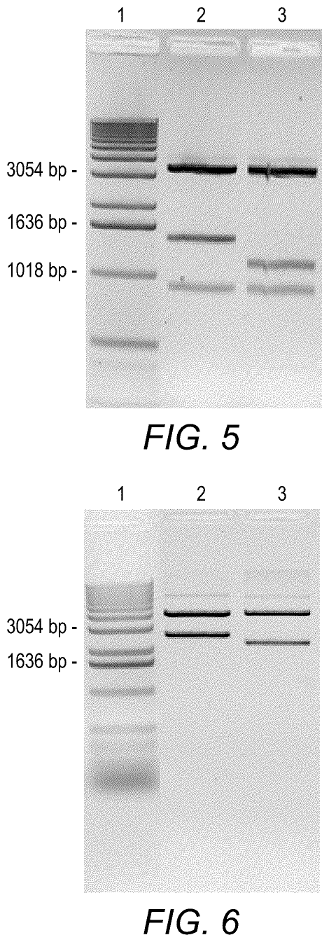

To generate recombinant scAAV viral particles, the cargo sequence (e.g., dominant negative RhoA) was inserted in the ITRs (wild-type and deleted, ITR-ΔITR) to create specially designed plasmids, named pGVB-2001-15 and pGVB-2001-16. The EF1α and CBh promoters were used in the pGVB-2001-15 and pGVB-2001-16 plasmids, respectively. The amino acid sequence of RhoA is shown in SEQ ID NO:6, with the dominant negative mutation being a T19N mutation shown in the dnRhoA sequence of SEQ ID NO:4. The dnRhoA containing plasmid was verified by restriction digestion with PvuII-HF, before going for the endotoxin-free maxi prep (FIG. 5). The integrity of ITR regions were verified by SmaI digestion (FIG. 6). Then, scAAV2 was produced by triple transfection of plasmids into HEK293 cells. Virus preps were purified by iodixanol gradient ultracentrifugation. The gradient was prepared by layering the following: 6 mL of 15% iodixanol, 6 mL of 25% iodixanol, 5 mL of 40% iodixanol and 5 mL of 54% iodixanol. 7-8 mL of lysate was added on top of the gradient and tubes were topped off with phosphate buffered saline (PBS).

The samples were centrifuged for 2 hours at 350,000×g at 18° C. After centrifugation, the tubes were punctured with a one-inch long and 18-gauge needle attached to a 10 mL syringe 3-5 mm below the 40/54 interface. Approximately 1 mL of the 54% interface was collected and 4 mL of the 40% interface was collected without disturbing the proteinaceous material at the 40/25 interface.

Following purification by ultracentrifugation, the viruses were concentrated and underwent a buffer exchange using Amicon ultra-15 50 kDa centrifugal filters. The filters were prepared by adding 15 mL of 0.1% Pluronic F-68 in PBS and incubated at room temperature for 10 minutes. Following the incubation, the 0.1% Pluronic solution was discarded. Then 15 mL of 0.01% Pluronic in PBS was added to the filter and centrifuged 2000× g for 5 minutes, the flowthrough was discarded. Finally, 15 mL of 0.001% Pluronic and 200 mM NaCl in PBS was added to the filter and centrifuged 2000×g for 5 minutes, the flowthrough was discarded. After the filter was prepared, the sample was diluted 1:2 with formulation buffer (0.001% Pluronic F-68 in PBS). The sample was added to the filter and centrifuged at 2000×g for 5 minutes. The flowthrough was discarded, and more samples can be added and centrifuged again. Once all the sample has been concentrated on the filter, formulation buffer was added, and centrifugation was repeated until 50 mL of formulation buffer has passed through the filter. Centrifugation was then continued in short intervals of 2-3 minutes until the desired volume is reached.

### Example 3: Using a Cell-Based Assay to Test the Function of scAAV2.CBh.dnRhoA

RhoA is a GTP-binding protein that cycles between an active and inactive form to activate ROCK. Overexpression of dnRhoA competes with endogenous RhoA to bind to ROCK, thus inhibiting the activation of ROCK. Measuring the activity of ROCK in cells can be developed as a cell-based assay to test the function of scAAV2.CBh.dnRhoA.

Members of the Rho family are essential regulatory components of the signaling pathway that directs cell motility, adhesion, and cytokinesis through reorganization of the action cytoskeleton. Rho is activated by extracellular signals such as lysophosphatidic acid (LPA). The actions of Rho are mediated by downstream Rho effectors. One of these effectors is ROCK. ROCK mediates Rho signaling and reorganizes the actin cytoskeleton through phosphorylation of several substrates that contribute to the assembly of actin filaments and contractility. For example, ROCK inactivates myosin phosphatase through the specific phosphorylation of myosin phosphatase target subunit 1 (MYPT1) at Thr696, which results in an increase in the phosphorylated content of the 20-kDa myosin light chain.

The ROCK Activity Assay Kit is an enzyme immunoassay developed for detection of the specific phosphorylation of MYPT1 at Thr696 by ROCK. A strip-well microtiter plate is precoated with a recombinant MYPT1. After incubating the substrate wells with ROCK samples, the phosphorylated MYPT1 is detected by an anti-phospho-MYPT1 (Thr696) antibody.

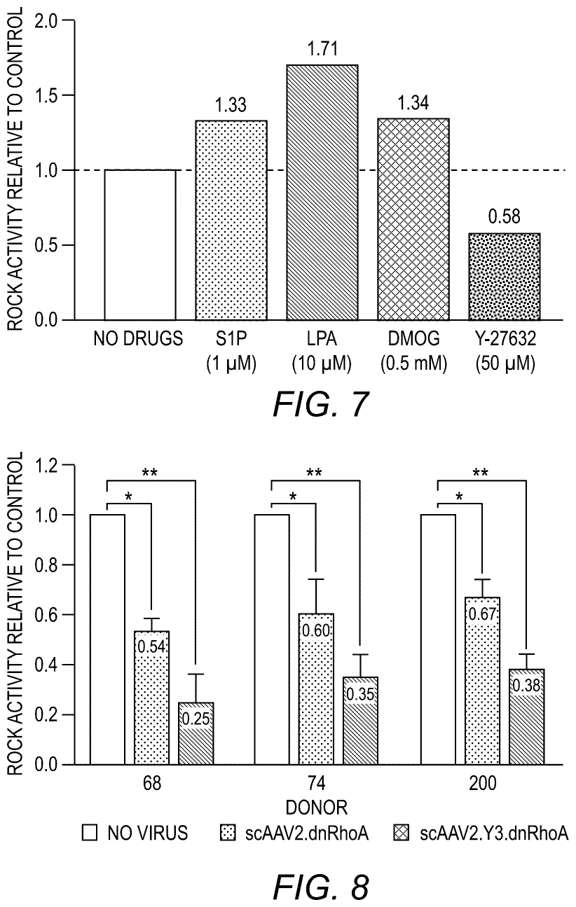

Human trabecular meshwork cells were seeded into 12-well plates at the density of 500,000 cells per well. 48 hours after seeding, cells were harvested with cell lysis buffer. Cell lysates were treated with ROCK activator or ROCK inhibitor before incubating with precoated strip-wells. The ROCK activity was then measured with the protocol of the kit. Three chemicals were used to activate ROCK: S1P (Sphingosine-1 phosphate at 1 μM), LPA (Oleoyl L-lysophosphatidic acid at 10 μM) and DMOG (Dimethyloxalylglycine at 0.5 mM). One chemical was used to inhibit ROCK activity: Y-27632 (50 μM). Data was presented as relative ROCK activity normalized to the no drug treated cell lysate. As shown in FIG. 7, all chemical activators increased the activity of ROCK, as LPA is the most potent activator. Chemical inhibitor decreased the ROCK activity as expected.

In the next experiment, human trabecular meshwork cells were seeded into 12-well plates at the density of 500,000 cells per well. Infections of scAAV2.Y3.CBh.dnRhoA or scAAV2.CBh.dnRhoA were performed at MOI 10,000 to compare the wild type AAV2 capsid and Y3 mutated AAV2 capsid. Cells were harvested 2 days after infection. ROCK activity was measured. The reduction of ROCK activity in cells infected by virus of Y3 mutated capsid was statistically significant, while the reduction in cells infected by virus of wild type capsid was less prominent (FIG. 8).

### Example 4: Measuring RhoA Expression in HTM Cells after Transduction of scAAV2.CBh.dnRhoA

In the next experiment, human trabecular meshwork cells were seeded into 12-well plates at the density of 500,000 cells per well. Infections of scAAV2.Y3.CBh.dnRhoA were performed at MOI 10,000. Cells were harvested 2 days after infection for total RNA extraction. RNA contents were measured by nano-drop. RT-PCR was performed to measure the expression of total RhoA.

### Example 5: Using a Transgene Model of Glaucoma to Test the Efficacy of scAAV2.CBh.dnRhoA

Efficacy of scAAV2.Y3.CBh.dnRhoA was tested in a rat model of glaucoma with elevated IOP obtained by Ad.BMP2 transduction. Elevated IOP is the result of an increased resistance of the trabecular meshwork tissue to aqueous humor outflow. This increased resistance can be caused by a variety of dysfunctional trabecular meshwork cells and mechanisms. It is widely accepted though, that the most common source of an increase in outflow resistance is the disruption of the organization of the trabecular meshwork's extracellular matrix (ECM). Bone morphogenetic protein 2 (BMP2) belongs to the superfamily of TGFβ proteins. BMP2 by itself has the full potential to initiate bone formation and to induce the differentiation of multipotent mesenchymal progenitor cells to the osteogenic lineage. Similarly, BMP2 induces osteogenic-like characteristics in primary HTM cells in vitro. Overexpression of the BMP2 gene in primary HTM cells transduced by an adenoviral vector increased alkaline phosphatase (ALP) activity, an enzyme that promote free phosphate and contributes to the formation of calcium phosphate precipitates (hydroxyapatite crystals), which are part of the mineralization process. Overexpression of BMP2 gene in the trabecular meshwork tissue would be sufficient to elevate IOP and created an animal model like ocular hypertension or glaucoma.

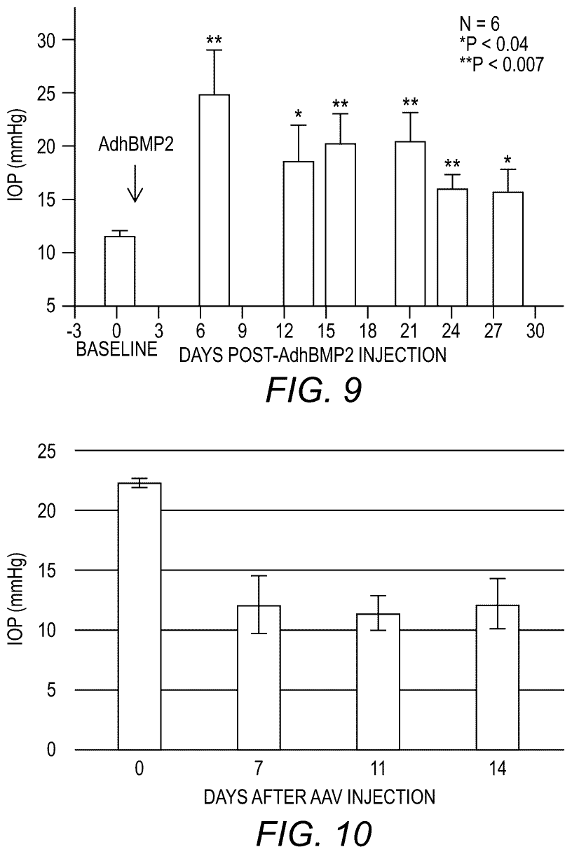

Intraocular pressure of rats at baseline was measured before Ad5.BMP2 injection, with a TonoLab tonometer (Icare). About 5 μl of Ad5.BMP2 virus (titer at 1.8×10¹⁰ pfu/ml) was injected in the anterior chamber of rat eyes under anesthesia. Then, IOP was monitored up to day 28 post-injection. The baseline IOP of rat eyes was about 12 mmHg. After injection of Ad5.BMP2, the average IOP quickly increased to about 25 mmHg, then stabilized around 15 mmHg (FIG. 9). At all time points of measurement, the IOP elevation was significant.

Once the IOP elevation was established, scAAV2.Y3.CBh.dnRhoA was injected to reduce the IOP. Only the eyes with more than 50% increase of IOP compared to the baseline were selected for the AAV injection. Then, IOP was continuously monitored up to 14 days post-AAV injections. As soon as 7 days after AAV injection, about 50% IOP reduction was observed. Then, the IOP was maintained at a level close to baseline up to day 14 (FIG. 10).

The foregoing is illustrative of the present invention, and is not to be construed as limiting thereof. The invention is defined by the following claims, with equivalents of the claims to be included therein.

1. A recombinant self-complementary adeno-associated virus (scAAV) particle comprising:
a) an AAV capsid protein; and
b) a scAAV viral genome comprising a eukaryotic promoter operably linked to a polynucleotide encoding a dominant negative RhoA.

2. The recombinant scAAV particle of claim 1, wherein the dominant negative RhoA comprises at least one amino acid mutation.

3. The recombinant scAAV particle of claim 2, wherein the at least one amino acid mutation is a threonine to asparagine mutation at amino acid position 19 (T19N) of SEQ ID NO:6.

4. The recombinant scAAV particle of claim 1, wherein the eukaryotic promoter is a short (e.g., about 100, 200, 300, 400, 500, 600, 700, 800, 900, 1000, 1100, or about 1200 nucleotides in length) eukaryotic promoter, optionally wherein the short eukaryotic promoter is truncated elongation factor 1-α (EF1α), a chicken beta-actin (CBA), or hybrid chicken beta-actin (CBh) promoter.

5. The recombinant scAAV particle of claim 1, wherein the polynucleotide encoding a dominant negative RhoA is codon optimized for expression in human (e.g., wherein the optimized particle expresses the polynucleotide encoding the dominant negative RhoA in human about 5%, 10%, 15%, 20%, 25%, 30%, 40%, 50%, 60%, 80%, 90%, 100%, 125%, 150%, 175%, 200%, 350%, 400%, 450%, or about 500% more than a non-optimized particle).

6. The recombinant scAAV particle of claim 1, wherein the capsid protein is optimized for expression in ocular tissues (e.g., wherein the optimized particle expresses the polynucleotide encoding the dominant negative RhoA in the ocular tissues about 5%, 10%, 15%, 20%, 25%, 30%, 40%, 50%, 60%, 80%, 90%, 100%, 125%, 150%, 175%, 200%, 350%, 400%, 450%, or about 500% more than a non-optimized particle).

7. The recombinant scAAV particle of claim 1, wherein the particle comprises an AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV12, AAV2R471A, bovine AAV, or mouse AAV serotype.

8. The recombinant scAAV particle of claim 1, wherein the capsid protein comprises one or more amino acid mutations.

9. The recombinant scAAV particle of claim 8, wherein the one or more amino acid mutations comprise a Y444F, Y500F, and/or Y730F amino acid mutation, numbered according to VP1 of AAV2 (SEQ ID NO: 5).

10. The recombinant scAAV particle of claim 8, wherein the one or more amino acid mutations reduce immunogenicity and/or increases expression of the dominant negative RhoA.

11. The recombinant scAAV particle of claim 6, wherein the ocular tissues comprise the trabecular meshwork, iris, cornea, and/or retina.

12. A method of reducing the intraocular pressure (TOP) in a subject in need thereof, the method comprising administering to the subject a therapeutically effective amount of the recombinant scAAV particle of claim 1, thereby reducing the IOP in the subject.

13. The method of claim 12, wherein the administering comprises intraocular injection.

14. The method of claim 13, wherein the intraocular injection comprises injection into the anterior chamber of the eye, optionally to the trabecular meshwork tissue and/or to the cornea, optionally an intracameral injection.

15. (canceled)

16. The method of claim 13, wherein the intraocular injection comprises injection into the posterior chamber of the eye, optionally to the retinal cells (e.g., the retinal ganglion cells and/or the retinal pigmented epithelial cells).

17. A method of treating and/or preventing an ocular disease in a subject in need thereof, the method comprising administering to the subject a therapeutically effective amount of the recombinant scAAV particle of claim 1, thereby treating and/or preventing the ocular disease in the subject.

18. The method of claim 17, wherein the ocular disease is associated with elevated TOP, e.g., glaucoma, age-related macular degeneration (AMD), diabetic retinopathy, and/or retinal holes.

19. (canceled)

20. The method of claim 17, wherein the administering comprises intraocular injection.

21. The method of claim 20, wherein the intraocular injection comprises injection into the anterior chamber of the eye, optionally to the trabecular meshwork tissue and/or to the cornea.

22. The method of claim 21, wherein the intraocular injection comprises injection into the posterior chamber of the eye, optionally to the retinal cells (e.g., the retinal ganglion cells and/or the retinal pigmented epithelial cells).


SEQUENCE LISTING (7 sequences)

SEQ ID NO: 1 - DNA, 1049 bp, synthetic construct
  source 1..1049: mol_type = other DNA
cgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggccggcttgagatccagctgttggggtgagtactccctctcaaaagcgggcatgacttctgcgctaagattgtcagtttccaaaaacgaggaggatttgatattcacctggcccgatctggccatacacttgagtgacaatgacatccactttgcctttctctccacaggtgtccactcccaggtccaa

SEQ ID NO: 2 - DNA, 1075 bp, synthetic construct
  source 1..1075: mol_type = other DNA
gaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttc

SEQ ID NO: 3 - DNA, 565 bp, synthetic construct
  source 1..565: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 4 - protein, 193 aa, synthetic construct
MAAIRKKLVIVGDGACGKNCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 5 - protein, 735 aa, Adeno-associated virus 2 [sequence omitted]

SEQ ID NO: 6 - protein, 193 aa, Homo sapiens
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 7 - DNA, 5721 bp, synthetic construct
  source 1..5721: mol_type = other DNA
ctggcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggagcaatggctgccatccggaagaaactggtgattgttggtgatggagcctgtggaaagaactgcttgctcatagtcttcagcaaggaccagttcccagaggtgtatgtgcccacagtgtttgagaactatgtggcagatatcgaggtggatggaaagcaggtagagttggctttgtgggacacagctgggcaggaagattatgatcgcctgaggcccctctcctacccagataccgatgttatactgatgtgtttttccatcgacagccctgatagtttagaaaacatcccagaaaagtggaccccagaagtcaagcatttctgtcccaacgtgcccatcatcctggttgggaataagaaggatcttcggaatgatgagcacacaaggcgggagctagccaagatgaagcaggagccggtgaaacctgaagaaggcagagatatggcaaacaggattggcgcttttgggtacatggagtgttcagcaaagaccaaagatggagtgagagaggtttttgaaatggctacgagagctgctctgcaagctagacgtgggaagaaaaaatctggttgccttgtcttgtgagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggaaccccactccctctctgcgcgctcgctcgctcactgaggccgggcgaccaaaggtcgcccgacgcccgggctttgcccgggcggcctcagtgagcgagcgagcgcgcagctgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacaggaaaaacgctcatggaaatacctacattttgacgctcaatcgtctggaattccattcgccattcaggctgcgcaactgttgggaagggcgatcggtgcgggcctcttcgctattacgccag